(19) United States
(12) Patent Application Publication
(10) Pub. No.: US 2005/0118178 A1
(43) Pub. Date: Jun. 2, 2005

(54) Use of t-cell immune response cdna7 (tirc7) in angiogenic and anti-angiogenic therapy

(76) Inventors: Nalan Utku, Berlin (DE); Peter Christian Warnke, Formby (GB)

Correspondence Address: John P White;Cooper & Dunham, 1185 Avenue of the Americas, New York, NY, 10036, US

(21) Appl. No.: 10/495,661

(22) PCT Filed: Nov. 27, 2002
(86) PCT No.: PCT/EP02/13384

(30) Foreign Application Priority Data
Nov. 27, 2001 (EP) 01 128 146.6

(51) Int. Cl. C12Q 1/68; G01N 33/574; A61K 48/00; A61K 39/395

(52) U.S. Cl. 424/155.1; 514/44; 435/6; 435/7.23

(57) ABSTRACT

A novel use of T-cell immune response cDNA 7 (TIRC7) in angiogenic and anti-angiogenic therapy is described. Provided are agents such as anti-TIRC7 antibodies and methods of treatment which through modulating the activity of TIRC7 can be used for enhancing or suppressing angiogenesis and/or neovascularization, an important feature in many pathological conditions including wound healing, solid tumors, developing metastases, ischemic heart diseases and diabetic retinopathy. Furthermore, diagnostic methods for the detection of such diseases using TIRC7 specific agents are provided.

The present invention relates generally to the use of T-cell immune response cDNA 7 (TIRC7), to the use of an agent which modulates the activity of TIRC7 and to the use of a nucleic acid molecule encoding said TIRC7 or said agent for the preparation of pharmaceutical compositions for enhancing or suppressing angiogenesis and/or neovascularization. In particular, the present invention relates to the use of TIRC7 or a fragment thereof, its encoding or regulatory nucleic acid sequences, to activators and antagonists of TIRC7 and to nucleic acid molecules encoding said activators or antagonists in angiogenic or anti-angiogenic therapy.

Several documents are cited throughout the text of this specification. Each of the documents cited herein (including any manufacturer's specifications, instructions, etc.) are hereby incorporated herein by reference; however, there is no admission that any document cited is indeed prior art as to the present invention.

Angiogenesis is a major feature in many pathological conditions including wound healing, solid tumors, developing metastases, ischemic heart diseases and diabetic retinopathy. While agents such as VEGF and other growth factors are presently being employed to stimulate the development of angiogenesis, for example, after arterial occlusion, there is a constant need for targets and agents capable of modulating angiogenesis and/or neovascularization. Furthermore, anti-angiogenic therapy has been proposed for the treatment not only of cancer but also of other diseases characterized by abnormal vasculature—such as hemangiomas, arteriovenous malformations, diabetic retinopathy and macular degeneration—for which anti-angiogenic approaches have already shown benefits (Folkman, J. in Harrison's Textbook of Internal Medicine, 15^{th} ed. (eds Braunwald, E. et al) 517-530 (McGraw-Hill, New York, 2001); Cameliet and Jain, Nature 407 (2000), 249-257; see also for review Jain, Nature Medicine 7 (2001), 987-989). Unfortunately, in direct and indirect anti-angiogenic therapies used so far, a second wave of angiogenesis ensues as the non-responsive or surviving cancer cells proliferate and produce angiogenic factors. The blood vessels of the relapsed tumor begin to exhibit abnormalities reminiscent of those of an untreated tumor. Thus, there is also need for targets and agents that can be efficiently used in anti-angiogenic therapy of tumors.

Thus, the technical problem of the present invention is to provide pharmaceutical compositions and methods for the modulation of angiogenesis and/or neovascularization. The solution to this technical problem is achieved by providing the embodiments characterized in the claims.

Accordingly, the invention relates generally to the use of T-cell immune response cDNA 7 (TIRC7), to the use of an agent, i.e. antagonist/inhibitor or agonist/activator, which modulates the activity of TIRC7 and to the use of a nucleic acid molecule encoding said TIRC7 or said agent for the preparation of pharmaceutical compositions for enhancing or suppressing angiogenesis and/or neovascularization.

Experiments aiming at studying expression of TIRC7 suggest that TIRC7 is highly expressed in proliferating capillary endothelial cells of tumors. It is therefore expected that TIRC7 has a potential in regulation of endothelial cell proliferation. Accordingly, it, is prudent to stipulate that TIRC7 and agonists thereof will be of benefit for stimulating angiogenesis and/or neovascularization which in turn can be used for the treatment of, for example, angiogenic diseases such as arterial occlusive diseases, like ischemic heart disease. Furthermore, experiments performed in accordance with the present invention demonstrated that an antibody against TIRC7 has an inhibitory effect on leukemic cell lines such as Jurkat and Sub T1. Since it could be shown that TIRC7 is highly expressed in proliferating capillary endothelial cells and such cells are needed to supply nutrition to tumors, TIRC7 has a potential in growth inhibition of endothelial cell proliferation and therefore an extremely important function in the treatment and diagnosis of angiogenesis in malignant tumors. Thus, it can be reasonably expected that anti-TIRC7 antibodies as well as other TIRC7 antagonists are able to significantly influence therapeutically, for example, the invasive growth of tumor cells, for example glioma. Accordingly, with TIRC7 the present invention provides a novel substance and target in angiogenic and anti-angiogenic therapy.

The term “TIRC7” as used in accordance with the present invention, denotes a protein which initially has been described to be involved in the signal transduction of T-cell activation and proliferation and that, preferably in a soluble form is capable of inhibiting or suppressing T-cell proliferation in response to alloactivation in a mixed lymphocyte culture or in response to mitogens when exogeneously added to the culture. In vitro translated TIRC7 protein has been shown to be able to efficiently suppress in a dose dependent manner the proliferation of T-cells in response to alloactivation in a mixed lymphocyte culture or in response to mitogens. TIRC7 is known to the person skilled in the art and described, inter alia, in WO99/11782, Utku, Immunity 9 (1998), 509-518 and Heinemann, Genomics 57 (1999), 398-406, which also disclose the amino and nucleic acid sequences of TIRC7.

The terms “antagonist/inhibitor and agonist/activator” in accordance with the present invention include chemical agents that modulate the action of TIRC7, either through altering its enzymatic or biological activity or through modulation of expression, e.g., by affecting transcription or translation. In some cases the antagonist/inhibitor or agonist/activator may also be a substrate or ligand binding molecule.

The term “activator,” as used herein, includes both substances necessary for TIRC7 to become active in the first place, and substances which merely accentuate its activity.

The term “inhibitor” includes both substances which reduce the activity of the TIRC7 and these which nullify it altogether. When more than one possible activity is defined herein for TIRC7, the inhibitor or activator may modulate any or all of TIRC7 activities. An “antagonist” or “agonist” that modulates the activity of TIRC7 and causes for example a response in a cell based assay refers to a compound that alters directly or indirectly the activity of TIRC7 or the amount of active TIRC7. Typically, the effect of an antagonist is substantially the same as that of the anti-TIRC7 antibodies described in Utku, Immunity 9 (1998), 509-518. Antagonists include competitive as well as non-competitive antagonists. A competitive antagonist (or competitive blocker) interacts with or near the site specific for agonist binding. A non-competitive antagonist or blocker inactivates the function of the receptor by interacting with a site other than the agonist interaction site. Preferably, the antagonist/inhibitor and agonist/activator of TIRC7 are small chemical agents which directly interact with TIRC7. Therefore, there will preferably be a direct relationship between the molar amount of compound required to inhibit or stimulate TIRC7 activity and the molar amount of TIRC7 present or lacking in the cell.

Activators and inhibitors may be designed by structure-assisted computer modeling for example according to alpha-helix and alpha-helix forming regions (“alpha-regions”), beta-sheet and beta-sheet-forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, flexible regions, surface-forming regions, substrate binding region, and high antigenic index regions.; Such preferred regions include Garnier-Robson alpha-regions, beta-regions, turn-regions, and coil-regions, Chou-Fasman alpha-regions, beta-regions, and turn-regions, Kyte-Doolittle hydrophilic regions and hydrophobic regions, Eisenberg alpha and beta amphipathic regions, Karplus-Schulz flexible regions, Emini surface-forming regions, and Jameson-Wolf high antigenic index regions. Computer predictions can be made made using for example GCG-software derived from HGMP resource center Cambridge (Rice, 1995) Programme Manual for the EGCG package. (Cambridge, CB10 IRQ, England: Hinxton Hall).

As mentioned above, in one aspect the, present invention relates to the use of T-cell immune response cDNA 7 (TIRC7), an activator of TIRC7 or of a nucleic acid molecule encoding said TIRC7 or said activator for the preparation of a pharmaceutical composition for enhancing angiogenesis and/or neovascularization. Said agonist/activator can be or can be derived from, for example, a TIRC7 polypeptide, a TIRC7 gene, an anti-TIRC7 antibody, a transcription regulator of the TIRC7 gene or a ligand binding molecule.

In a preferred embodiment, the use of the invention may be employed for the treatment of diseases caused by a vascular disease or a cardiac infarct or a stroke or for the treatment of any disease where an increase of blood supply via collaterals, arteries etc. is needed. Preferably, said pharmaceutical composition is applied to a subject suffering from a vascular disease or a cardiac infarct or a stroke.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease. i.e. arresting its development; or (c) relieving the disease, i.e. causing regression of the disease.

Furthermore, the term “subject” as employed herein relates to animals in need of amelioration, treatment and/or prevention of angiogenic and vascular diseases including malignant tumors as disclosed herein. Most preferably said subject is a human.

In a particularly preferred embodiment, the methods and uses of the invention are applied to treat a subject suffering from arteriosclerosis, a coronary artery disease, a cerebral occlusive disease, a peripheral occlusive disease, a visceral occlusive disease, renal occlusive disease, a mesenterial arterial insufficiency or an ophthamic or retenal occlusion or for any disease where atherosclerotic plaques in the vascular wall lead to an obstruction of the vessel diameter.

In a further preferred embodiment, the methods and uses of the invention are applied to a subject during or after exposure to an agent or radiation or surgical treatment which damage or destroy arteries.

TIRC7 used in the methods and uses of the invention is typically a recombinant TIRC7 or expressed by a recombinant DNA molecule in a recipient cell of the subject to be treated. DNA sequences encoding TIRC7 as well as functional derivatives and functionally equivalent substances which can be used in the methods and uses of the invention are described in the prior art; see the references cited above. Moreover. DNA and amino acid sequences of TIRC7 are available in the Gene Bank database. As described above, methods for the production of recombinant proteins are well-known to the person skilled in the art; see, e.g., Sambrook, Molecular Cloning A Laboratory Manual, Cold Spring Harbor Laboratory (1989) N.Y. and Ausubel. Current Protocols in Molecular Biology, Green Publishing Associates and Wiley Interscience, N.Y. (1989), (1994).

In a further preferred embodiment, the pharmaceutical composition in the method and the use of the present invention is applied in conjugation with a growth factor, preferably fibroblast growth factor (FGF) or vascular endothelial growth factor (VEGF). This embodiment is particularly suited for enhancing of both sprouting of capillaries (angiogenesis) and in situ enlargement of preexisting arteriolar connections into true collateral arteries. Pharmaceutical compositions comprising, for example, TIRC7, and a growth factor such as VEGF may be used for the treatment of peripheral vascular diseases or coronary artery disease.

It is envisaged by the present invention that TIRC7 and the nucleic acid molecules encoding TIRC7 or entities of the corresponding activator are administered either alone or in combination, and optionally together with a pharmaceutically acceptable carrier or exipient. Said nucleic acid molecules may be stably integrated into the genome of the cell or may be maintained in a form extrachromosomally, see, e.g., Calos, Trends Genet. 12 (1996), 463-466. On the other hand, viral vectors described in the prior art may be used for transfecting certain cells, tissues or organs. Furthermore, it is possible to use a pharmaceutical composition of the invention which comprises a nucleic acid molecule encoding a TIRC7 in gene therapy. Suitable gene delivery systems may include liposomes, receptor-mediated delivery systems, naked DNA, and viral vectors such as herpes viruses, retroviruses, adenoviruses, and adeno-associated viruses, among others. Delivery of nucleic acid molecules to a specific site in the body for gene therapy may also be accomplished using a biolistic delivery system, such as that described by Williams (Proc. Natl. Acad. Sci. USA 88 (1991), 2726-2729).

Standard methods for transfecting cells with nucleic acid molecules are well known to those skilled in the art of molecular biology, see, e.g., WO 94/29469. Gene therapy to prevent or decrease the development of diseases described herein may be carried out by directly administering the nucleic acid molecule encoding TIRC7 to a patient or by transfecting cells with said nucleic acid molecule ex vivo and infusing the transfected cells into the patient. Furthermore, research pertaining to gene transfer into cells of the germ line is one of the fastest growing fields in reproductive biology. Gene therapy, which is based on introducing therapeutic genes into cells by ex-vivo or in-vivo techniques is one of the most important applications of gene transfer. Suitable vectors and methods for in-vitro or in-vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813; Isner, Lancet 348 (1996), 370-374; Muhlhauser, Circ. Res. 77 (1995), 1077-1086; Wang, Nature Medicine 2 (1996), 714-716; WO94/29469; WO 97/00957 or Schaper, Current Opinion in Biotechnology 7 (1996), 635-640, and references cited therein. The nucleic acid molecules comprised in the pharmaceutical composition of the invention may be designed for direct introduction or for introduction via liposomes, or viral vectors (e.g. adenoviral, retroviral) containing said nucleic acid molecule into the cell. Preferably, said cell is a germ line cell, embryonic cell, or egg cell or derived therefrom.

Thus, in a preferred embodiment: the nucleic acid molecule comprised in the pharmaceutical composition for the use of the invention is designed for the expression of TIRC7 by cells in vivo by, for example, direct introduction of said nucleic acid molecule or introduction of a corresponding plasmid, a plasmid in liposomes, or a viral vector (e.g. adenoviral, retroviral) containing said nucleic acid molecule. Particularly suitable vectors for use in angiogenic and anti-angiogenic therapy are described in, e.g. Fathallah-Shaykh, J. Immunol. 164 (2000), 217-222 and Varda-Bloom, Gene Therapy 8 (2001), 819-827.

In this context, it is understood that TIRC7 to be employed according to the present invention may be, e.g., modified by conventional methods known in the art. For example, it is possible to use fragments which retain the biological activity of TIRC7, namely the capability of promoting endothelial cell growth. This further allows the construction of chimeric proteins and peptides wherein other functional amino acid sequences may be either physically linked by, e.g., chemical means to TIRC7 or may be fused by recombinant DNA techniques well known in the art. Furthermore, folding simulations and computer redesign of structural motifs of the TIRC7 or its ligands can be performed using appropriate computer programs (Olszewski, Proteins 25 (1996), 286-299; Hoffman, Comput. Appl. Biosci. 11 (1995), 675-679). Computer modeling of protein folding can be used for the conformational and energetic analysis of detailed receptor and protein models (Monge, J. Mol. Biol. 247 (1995), 995-1012; Renouf, Adv. Exp. Med. Biol. 376 (1995), 37-45). In particular, the appropriate programs can be used for the identification of interactive sites of TIRC7 and its ligand by computer assistant searches for complementary peptide sequences (Fassina, Immunomethods 5 (1994), 114-120). Further appropriate computer systems for the design of protein and peptides are described in the prior art, for example in Berry, Biochem. Soc. Trans. 22 (1994), 1033-1036; Wodak, Ann. N. Y. Acad. Sci. 501 (1987), 1-13; Pabo, Biochemistry 25 (1986), 5987-5991. The results obtained from the above-described computer analysis can be used for, e.g., the preparation of peptide mimetics of TIRC7.

As discussed above, neovascularization and the growth of arteries from preexisting arteriolar connections is essential for the delivery of nutrition to tumors. Thus, if the growth of said vessels to the tumor would be suppressed suppression and/or inhibition of tumor growth is to be expected. Selectively, it was possible to stain in fresh-frozen tissue, and to a lesser extent in paraffin embedded tissue, capillary endothelial cells which showed a very high expression of the TIRC7 antigen in the cell membranes. It is very likely, that proliferation experiments by using different escalating doses of antibody against TIRC7 will influence the capacity for confluent growth also by using co-cultures with normal brain or chicken brain or fibroblasts. This approach has a therapeutic potential to treat brain tumors and other non brain tumors and there metastasis. Indeed, by performing experiments in order to block tumor cell growth, it could for example be shown that antibody against TIRC7 has an inhibitory effect on leukemic cell lines such as Jurkat and Sub T1. Thus, it can be reasonably expected that anti-TIRC7 antibodies as well as other TIRC7 antagonists are able to significantly influence therapeutically, for example, the invasive growth of the glioma as well as other tumor cells. Besides that the highly selective staining of proliferating capillary endothelial cells renders a new target for TIRC7, it might have a potential in growth inhibition of endothelial cell proliferation and therefore extremely important function in treatment and diagnosis of neoangiogenesis in malignant brain tumors or other solid extra cerebral brain tumors.

Accordingly, in a further aspect the present invention relates to the use of an antagonist of T-cell immune response cDNA 7 (TIRC7) or of a nucleic acid molecule encoding said antagonist for the preparation of a pharmaceutical composition for suppressing angiogenesis and/or neovascularization.

TIRC7 antagonists which suppress angiogenesis and/or neovascularization may be peptides, proteins, nucleic acids, antibodies, small organic compounds, peptide mimics, aptamers or PNAs (Milner, Nature Medicine 1 (1995), 879-880; Hupp, Cell 83 (1995), 237-245; Gibbs, Cell 79 (1994), 193-198; Gold, Ann. Rev. Biochem. 64 (1995), 736-797). For the preparation and application of such compounds, the person skilled in the art can use the methods known in the art, for example those referred to above. Furthermore, antagonists/inhibitors of TIRC7 and methods for obtaining the same are described in, for example, PCT/EP01/12485.

In a preferred embodiment, said pharmaceutical composition is applied to a subject suffering from a malignant tumor. Preferably, said tumor is an intra cerebral brain tumor or a solid extra cerebral brain tumor.

In a further preferred embodiment of the use of the present invention, the antagonist blocks an interaction of TIRC7 and its ligand. In this embodiment the ligand is usually a TIRC7 receptor on malignant tumor cells or on endothelial cells-in malignant tumors.

The TIRC7 antagonist can be or comprise an antibody, a (poly)peptide, a nucleic acid molecule, a small organic compound, a TIRC7 ligand, peptide nucleic acid (PNA), aptamer, or peptide mimetic.

Nucleic acid molecules specifically hybridizing to TIRC7 encoding genes and/or their regulatory sequences may be used for repression of expression of said gene, for example due to an antisense or triple helix effect or they may be used for the construction of appropriate ribozymes (see, e.g., EP-B1 0 291 533, EP-A1 0 321 201, EP-A2 0 360 257) which specifically cleave the (pre)-mRNA of a gene encoding TIRC7. The nucleic acid sequence encoding TIRC7 is known in the art; see references supra. Selection of appropriate target sites and corresponding ribozymes can be done as described for example in Steinecke, Ribozymes, Methods in Cell Biology 50, Galbraith et al. eds Academic Press, Inc. (1995), 449-460. Furthermore, methods are described in the literature for identifying nucleic acid molecules such as an RNA fragment that mimics the structure of a defined or undefined target RNA molecule to which a compound binds inside of a cell resulting in retardation of cell growth or cell death; see, e.g., WO 98/18947 and references cited therein. These nucleic acid molecules can be used for identifying unknown compounds of pharmaceutical interest, and for identifying unknown RNA targets for use in treating a disease. Alternatively, for example, the conformational structure of the RENA fragment which mimics the binding site can be employed in rational drug design to modify known ligands to make them bind more avidly to the target. One such methodology is nuclear magnetic resonance (MR), which is useful to identify drug and RNA conformational structures. Still other methods are, for example, the drug design methods as described in WO 95/35367, U.S. Pat. No. 5,322,933, where the crystal structure of the RNA fragment can be deduced and computer programs are utilized to design novel binding compounds which can act as antibiotics.

Nucleic acid sequences that are complementary to the TIRC7 encoding gene sequence or sense nucleic acid sequences can be synthesized for antisense therapy. These sense or antisense molecules may be DNA, stable derivatives of DNA such as phosphorothioates or methylphosphonates, RNA, stable derivatives of RNA such as 2′-O-alkylRNA, or other TIRC7 antisense oligonucleotide mimetics. TIRC7 antisense molecules may be introduced into cells by microinjection, liposome encapsulation or by expression from vectors harboring the antisense sequence. TIRC7 antisense therapy may be particularly useful for the treatment of diseases where it is beneficial to reduce TIRC7 activity. TIRC7 gene therapy may be used to introduce TIRC7 into the cells of target organisms. The TIRC7 gene can be ligated into viral vectors that mediate transfer of the TIRC7 DNA by infection of recipient host cells. Suitable viral vectors include retrovirus, adenovirus, adeno-associated virus, herpes virus, vaccinia virus, polio virus and the like. Alternatively, TIRC7 DNA can be transferred into cells for gene therapy by non-viral techniques including receptor-mediated targeted DNA transfer using ligand-DNA conjugates or adenovirus-ligand-DNA conjugates, lipofection membrane fusion or direct microinjection. These procedures and variations thereof are suitable for ex vivo as well as in vivo TIRC7 gene therapy. TIRC7 gene therapy may be particularly useful for the treatment of diseases where it is beneficial to elevate TIRC7 activity. Protocols for molecular methodology of gene therapy suitable for use with the TIRC7 gene is described in Gene Therapy Protocols, edited by Paul D. Robbins, Human press, Totawa N.J., 1996.

Furthermore, the so-called “peptide nucleic acid” (PNA) technique can be used for the inhibition of the expression of a gene encoding a TIRC7. For example, the binding of PNAs to complementary as well as various single stranded RNA and DNA nucleic acid molecules can be systematically investigated using, e.g., thermal denaturation and BIAcore surface-interaction techniques (Jensen, Biochemistry 36 (1997), 5072-5077). The synthesis of PNAs can be performed according to methods known in the art, for example, as described in Koch, J. Pept. Res. 49 (1997), 80-88, Finn, Nucleic Acids Research 24 (1996), 3357-3363. Furthermore, folding simulations and computer redesign of structural motifs of TIRC7 and its receptors or ligands can be performed as described above to design drugs capable of inhibiting the biological activity of TIRC7.

Preferably, antibodies can be employed in accordance with the present invention specifically recognizing TIRC7 or their receptors or parts, i.e. specific fragments or epitopes, of such TIRC7s and ligands thereby inactivating the TIRC7 or the TIRC7 ligand. These antibodies can be monoclonal antibodies, polyclonal antibodies or synthetic antibodies as well as fragments of antibodies, such as Fab, Fv or scFv fragments etc. Antibodies or fragments thereof can be obtained by using methods which are described, e.g., in Harlow and Lane “Antibodies, A Laboratory Manual”, CSH Press, Cold Spring Harbor, 1988 or EP-B1 0 451 216 and references cited therein. For example, surface plasmon resonance as employed in the BIAcore system can be used to increase the efficiency of phage antibodies which bind to an epitope of the TIRC7 or its ligand (Schier, Human Antibodies Hybridomas 7 (1996), 97-105; Malmborg, J. Immunol. Methods 183 (1995), 7-13).

Putative inhibitors which can be used in accordance with the present invention including peptides, proteins, nucleic acids, antibodies, small organic compounds, ligands, hormones, peptide mimetics, PNAs and the like capable of inhibiting the biological activity of TIRC7 or its ligand may be identified according to the methods known in the art, for example as described in EP-A-0 403 506.

In a preferred embodiment of the present invention, the antagonist is a nucleic acid molecule and designed to be expressed in vascular cells or cells surrounding preexisting arteriolar connections to a tumor.

In a particularly preferred embodiment of the methods and uses of the invention the antagonist is
(i) an anti-TIRC7 antibody or an anti-TIRC7-ligand antibody; and/or
(ii) a non-stimulatory form of TIRC7 or a soluble form of a-TIRC7-ligand.

An anti-TIRC7 antibody to be used in accordance with pharmaceutical compositions of the present invention can be preferably a monoclonal antibody, but also include a polyclonal antibody, a single chain antibody, human or humanized antibody, primatized, chimerized or fragment thereof that specifically birds TIRC7 peptide or polypeptide also including bispecific antibody, synthetic antibody, antibody fragment, such as Fab, Fv or scFv fragments etc., or a chemically modified derivative of any of these. The general methodology for producing antibodies is well-known and has been described in, for example, Köhler and Milstein, Nature 256 (1975), 494 and reviewed in J. G. R. Hurrel, ed., “Monoclonal Hybridoma Antibodies: Techniques and Applications”, CRC Press Inc., Boco Raron, Fla. (1982), as well as that taught by L. T. Miruns et al., Virology 176 (1990), 604-619.

Further sources for the basic structure of inhibitors can be employed and comprise, for example, mimetic analogs of the TIRC7 polypeptide. Mimetic analogs of the TIRC7 polypeptide can be generated by, for example, substituting the amino acids that are expected to be essential for the biological activity with, e.g., stereoisomers, i.e. D-amino acids; see e.g., Tsukida. J. Med. Chem. 40 (1997), 3534-3541. Furthermore, the TIRC7 polypeptide can be used to identify synthetic chemical peptide mimetics that bind to or can function as a ligand, substrate, binding partner or the receptor of the TIRC7 polypeptide as effectively as does the natural polypeptide; see, e.g., Engleman. J. Clin. Invest. 99 (1997), 2284-2292. For example, folding simulations and computer redesign of structural motifs of the protein of the invention can be performed using appropriate computer programs (Olszewski, Proteins 25 (1996), 286-299; Hoffman, Comput. Appl. Biosci. 11 (1995), 675-679), Computer modeling of protein folding can be used for the conformational and energetic analysis of detailed peptide and protein models (Monge, J. Mol. Biol. 247 (1995), 995-1012; Renouf, Adv. Exp. Med. Biol. 376 (1995), 37-45). In particular, the appropriate programs can be used for the identification of interactive sites of the TIRC7 polypeptide and its ligand or other interacting proteins by computer assistant searches for complementary peptide sequences (Fassina, Immunomethods 5 (1994), 114-120. Further appropriate computer systems for the design of protein and peptides are described in the prior art, for example in Berry, Biochem. Soc. Trans. 22 (1994), 1033-1036; Wodak, Ann. N. Y. Acad. Sci. 501 (1987), 1-13; Pabo, Biochemistry 25 (1986), 5987-5991. Methods for the generation and use of peptide mimetic combinatorial libraries are described in the prior art, for example in Ostresh, Methods in Enzymology 267 (1996), 220-234 and Dorner, Bioorg. Med. Chem. 4 (1996), 709-715. Furthermore, a three-dimensional and/or crystallographic structure of the TIRC7 protein can be used for the design of mimetic inhibitors of the biological activity of the protein of the invention (Rose, Biochemistry 35 (1996), 12933-12944; Rutenber, Bioorg. Med. Chem. 4 (1996), 1545-1558).

It is also well known to the person skilled in the art, that it is possible to design, synthesize and evaluate mimetics of small organic compounds that, for example, can act as a substrate or ligand to the TIRC7 polypeptide. For example, it has been described that D-glucose mimetics of hapalosin exhibited similar efficiency as hapalosin in antagonizing multidrug resistance assistance-associated protein in cytotoxicity; see Dinh, J. Med. Chem. 41 (1998), 981-987.

Recombinant TIRC7 polynucleotides, antisense molecules, vectors incorporating such polynucleotides or antisense molecules can be produced by methods known to those skilled in molecular biology. For example, the choice of vectors which would depend on the function desired and include plasmids, cosmids, viruses, bacteriophages and other vectors used conventionally in genetic engineering. Methods which are well known to those skilled in the art can be used to construct various plasmids and vectors; see, for example, the techniques described in Sambrook, and Ausubel cited supra. Alternatively, the polynucleotides and vectors can be reconstituted into liposomes for delivery to target cells. Relevant sequences can be transferred into expression vectors where expression of a particular polypeptide is required. Typical cloning vectors include pBscpt sk, pGEM, pUC9, pBR322 and pGBT9. Typical expression vectors include pTRE, pCAL-n-EK, pESP-1, pOP13CAT, pET, pGEX, pMALC, pPIC9, pBac.

The antibodies, nucleic acid molecules, inhibitors and activators used in the compositions of the present invention preferably have a specificity at least substantially identical to the binding specificity of the natural ligand or binding partner of the TIRC7 protein, in particular if TIRC7 stimulation is desired. An antibody or inhibitor can have a binding affinity to the TIRC7 protein of at least 10⁵ M⁻¹, preferably higher than 10⁷ M⁻¹ and advantageously up to 10¹⁰M⁻¹ in case TIRC7 suppression should be mediated.

In a preferred embodiment, a suppressive antibody or inhibitor has an affinity of at least about 10⁻⁷ M, preferably at least about 10⁻⁹ M and most preferably at least about 10⁻¹¹ M; and a TIRC7 stimulating activator has an affinity of less than about 10⁻⁷ M, preferably less than about 10⁻⁶ M and most preferably in order of 10⁻⁵M.

In case of antisense nucleic acid molecules it is preferred that they have a binding affinity to those encoding the TIRC7 protein of at most 2-, 5- or 10-fold less than an exact complement of 20 consecutive nucleotides of the coding sequence.

In a preferred embodiment of the invention, said pharmaceutical composition comprising an antagonist of TIRC7 or a nucleic acid molecule encoding said antagonist is used for the treatment of a tumor selected from a group consisting of glioblastoma, medulloblastoma, astrocytoma, primitive neuroectoderma, brain stem glioma cancers, colon carcinoma, bronchial carcinoma, sarcoma, carcinoma in the breast, carcinoma in the head/neck, mesothelioma, leukemia and meningeoma.

In accordance with the above, the present invention also relates to the use of a TIRC7 polypeptide or a biologically active fragment thereof, a nucleic acid molecule encoding TIRC7 or a nucleic acid molecule of at least 15 nucleotides in length hybridizing to a TIRC7 gene, an anti-TIRC7 antibody or of an TIRC7 activity assay for a method of obtaining, identifying and/or profiling a drug candidate for therapy of a vascular disorder as described above.

Pharmaceutically useful compositions such as described herein-before, comprising TIRC7 DNA, TIRC7 RNA, or TIRC7 protein, or modulators of TIRC7 activity, i.e. activator/agonist or inhibitor/antagonist, or chemical derivatives thereof may be formulated according to known methods such as by the admixture of a pharmaceutically acceptable carrier. Examples of such carriers and methods of formulation may be found in Remington's Pharmaceutical Sciences. To form a pharmaceutically acceptable composition suitable for effective administration, such compositions will contain an effective amount of the protein, DNA, RNA, or modulator.

Therapeutic or diagnostic compositions of the invention are administered to an individual in amounts sufficient to treat or diagnose disorders in which modulation of TIRC7-related activity is indicated. The effective amount may vary according to a variety of factors such as the individual's condition, weight, sex and age. Other factors include the mode of administration. The pharmaceutical compositions may be provided to the individual by a variety of routes such as by intracoronary, intraperitoneal, subcutaneous, intravenous, transdermal, intrasynovial, intramuscular or oral routes.

The term “chemical derivative” describes a molecule that contains additional chemical moieties that are not normally a part of the base molecule. Such moieties may improve the solubility, half-life, absorption, etc. of the base molecule. Alternatively the moieties may attenuate undesirable side effects of the base molecule or decrease the toxicity of the base molecule. Examples of such moieties are described in a variety of texts, such as Remington's Pharmaceutical Sciences.

TIRC7 DNA, TIRC7 RNA, or TIRC7 protein, or modulators of TIRC7 activity disclosed herein may be used alone at appropriate dosages defined by routine testing in order to obtain optimal activation or inhibition of the TIRC7 activity while minimizing any potential toxicity. In addition, co-administration or sequential administration of other agents may be desirable.

A therapeutically effective dose refers to that amount of protein, antibodies, nucleic acid, agonists, activators, antagonists, or inhibitors which ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

The present invention also has the objective of providing suitable topical, oral, systemic and parenteral pharmaceutical formulations for use in the novel methods of treatment of the present invention. The compositions containing compounds or modulators identified according to this invention as the active ingredient for use in the modulation of TIRC7 can be administered in a wide variety of therapeutic dosage forms in conventional vehicles for administration. For example, the compounds or modulators can be administered in such oral dosage forms as tablets, capsules (each including timed release and sustained release formulations), pills, powders, granules, elixirs, tinctures, solutions, suspensions, syrups and emulsions, or by injection. Likewise, they may also be administered in intravenous (both bolus and infusion), intraperitoneal, subcutaneous, topical with or without occlusion, or intramuscular form, all using forms, well known to those of ordinary skill in the pharmaceutical arts. An effective but non-toxic amount of the compound desired can be employed as a TIRC7 modulating agent.

The daily dosage of the products may be varied over a wide range from 0.01 to 1,000 mg per patient, per day. For oral administration, the compositions are preferably provided in the form of scored or unscored tablets containing 0.01, 0.05, 0.1, 0.5, 1.0, 2.5, 5.0, 10.0, 15.0, 25.0, and 50.0 milligrams of the active ingredient for the symptomatic adjustment of the dosage to the patient to be treated. An effective amount of the drug is ordinarily supplied at a dosage level of from about 0.0001 mg/kg to about 100 mg/kg of body weight per day. The range is more particularly from about 0.001 mg/kg to 10 mg/kg of body weight per day. The dosages of the TIRC7 modulators are adjusted when combined to achieve desired effects. On the other hand, dosages of these various agents may be independently optimized and combined to achieve a synergistic result wherein the pathology is reduced more than it would be if either agent were used alone.

A physician or veterinarian of ordinary skill can readily determine and prescribe the effective amount of the drug required to prevent, counter or arrest the progress of the condition. Optimal precision in achieving concentrations of drug within the range that yields efficacy without toxicity requires a regimen based on the kinetics of the drug's availability to target sites. This involves a consideration of the distribution, equilibrium, and elimination of a drug. In the methods of the present invention, the compounds or modulators herein described in detail can form the active ingredient, and are tropically administered in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices. For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

For liquid forms the active drug component can be combined in suitably flavored suspending or dispersing agents such as the synthetic and natural gums, for example, tragacanth, acacia, methyl-cellulose and the like. Other dispersing agents that may be employed include glycerin and the like. For parenteral administration, sterile suspensions and solutions are desired. Isotonic preparations, which generally contain suitable preservatives, are employed when intravenous administration is desired.

Topical preparations containing the active drug component can be admixed with a variety of carrier materials well known in the art, such as, e.g., alcohols, aloe vera gel, allantoin, glycerine, vitamin A and E oils, mineral oil, PPG2 myristyl propionate, and the like, to form, e.g., alcoholic solutions, topical cleansers, cleansing creams, skin gels, skin lotions, and shampoos in cream or gel formulations.

Based on these findings of the present invention, TIRC7 clearly allows to selectively target tumor cells within the brain, especially proliferating capillary cells, i.e. areas of neovascularisation, which are associated with a malignant phenotype in gliomas. Also clear is that there is no antigen expression in low-grade gliomas. The findings, especially that TIRC7 is actively expressed in endothelial cells in malignant tumors such as gliomas as well as in malignant tumor cells per se, offers specific targeting of these cells with molecules linked to TIRC7 and might be superior to the currently used targets for specific immunotherapy, which are the transferrin receptor (Shin, Proc. Natl. Acad. Sci. USA 92 (1995), 2820-2824) and the IL4 receptor of tumor cells. Thus, anti-TIRC7 antibody or a binding fragment thereof can be fused to for example a toxin or to a label if tumor imaging is desired. The fusion can be done by a variety of means, for example physical-linking or recombinant DNA-technology. Such methods are known in the art. For example, a recombinant fusion toxin targeting HER-2/NEU-over-expressing tumor cells and containing human tumor necrosis factor has been constructed by fusing cDNA for the single-chain recombinant antibody sFv23 recognizing the cell-surface domain of HER2/neu to the cDNA encoding human TNF; see Hombach, Int. J. Cancer 88 (2000), 267-273. A similar approach can be used in accordance with the present invention using cDNA of an anti-TIRC7 antibody instead of the anti-HER2/neu antibody. Further such approaches are described in the literature (see e.g. Rosenblum, Gastroenterology 113 (1997), 1163-1170) and can be easily adapted to methods and uses of the present invention.

Accordingly, in a further aspect the present invention relates to the use of anti-TIRC7 antibody or equivalent TIRC7 binding molecule for targeting malignant tumor cells or endothelial cells in malignant tumors. Preferably, said tumor is a tumor as defined herein above.

Further means and methods which can be employed and adapted in accordance with the uses and methods of the present invention regarding inhibiting tumor growth are described in for example WO00/44404, hereby incorporated by reference, where integrin is described as the target molecule to treat tumors in the brain.

In a further embodiment the present invention relates to a method of diagnosing a metastatic disease in a subject comprising:
a) assaying a sample from a subject for TIRC7 transcriptional activity; and
b) determining the existence of metastatic disease characterized by the induction of TIRC7 transcriptional activity.

In a still further embodiment the present invention relates to a method of diagnosing a metastatic disease in a subject comprising:
a) assaying a sample from a subject for the presence of TIRC7 protein; and,
b) determining the existence of metastatic disease by the presence of TIRC7 protein, wherein the abnormal presence of TIRC7 protein indicates the presence of a metastatic disease.

In another embodiment, the present invention relates to a method of diagnosing arteriosclerosis, a coronary artery disease, a cerebral occlusive disease, a peripheral occlusive disease, a visceral occlusive disease, renal occlusive disease, a mesenterial arterial insufficiency or an ophthamic or retenal occlusion or for any disease where atherosclerotic plaques in the vascular wall lead to an obstruction of the vessel diameter, said method comprising:
a) assaying a sample from a subject for TIRC7 transcriptional activity or TIRC7 protein; and
b) determining the existence of any one of the mentioned diseases, wherein the lack of induction of TIRC7 transcriptional activity or the abnormal absence of TIRC7 protein indicates the presence of the diseases.

In these embodiments, the TIRC7 polynucleotides, nucleic acid molecules, (poly)peptide, antibodies or ligands preferably detectably labeled. A variety of techniques are available for labeling biomolecules, are well known to the person skilled in the art and are considered to be within the scope of the present invention. Such techniques are, e.g., described in Tijssen, “Practice and theory of enzyme immuno assays” , Burden, R H and von Knippenburg (Eds), Volume 15 (1985), “Basic methods in molecular biology”; Davis L G, Dibmer M D; Battey Elsevier (1990), Mayer et al., (Eds) “Immunochemical methods in cell and molecular biology” Academic Press, London (1987), or in the series “Methods in Enzymology”, Academic Press, Inc. There are many different labels and methods of labeling known to those of ordinary skill in the art. Commonly used labels comprise, inter alia, fluorochromes (like fluorescein, rhodamine, Texas Red, etc.), enzymes (like horse radish peroxidase, β-galactosidase, alkaline phosphatase), radioactive isotopes (like ³²P or ¹²⁵I), biotin, digoxygenin, colloidal metals, chemi- or bioluminescent compounds (like dioxetanes, luminol or acridiniums). Labeling procedures, like covalent coupling of enzymes or biotinyl groups, iodinations, phosphorylations, biotinylations, random priming, nick-translations, tailing (using terminal transferases) are well known in the art. Detection methods comprise, but are not limited to, autoradiography, fluorescence microscopy, direct and indirect enzymatic reactions, etc.

In addition, the above-described compounds etc. may be attached to a solid phase. Solid phases are known to those in the art and may comprise polystyrene beads, latex beads, magnetic beads, colloid metal particles, glass and/or silicon chips and surfaces, nitrocellulose strips, membranes, sheets, animal red blood cells, or red blood cell ghosts, duracytes and the walls of wells of a reaction tray, plastic tubes or other test tubes. Suitable methods of immobilizing TIRC7 nucleic acids, (poly)peptides, proteins, antibodies, etc. on solid phases include but are not limited to ionic, hydrophobic, covalent interactions and the like. The solid phase can retain one or more additional receptor(s) which has/have the ability to attract and immobilize the region as defined above. This receptor can comprise a charged substance that is oppositely charged with respect to the reagent itself or to a charged substance conjugated to the capture reagent or the receptor can be any specific binding partner which is immobilized upon (attached to) the solid phase and which is able to immobilize the reagent as defined above.

Commonly used detection assays can comprise radioisotopic or non-radioisotopic methods. These comprise, inter alia, RIA (Radioisotopic Assay) and IRMA (Immune Radioimmunometric Assay), EIA (Enzym Immuno Assay), ELISA (Enzyme Linked Immuno Assay), FIA (Fluorescent Immuno Assay), and CLIA (Chemioluminescent Immune Assay). Other detection methods that are used in the art are those that do not utilize tracer molecules. One prototype of these methods is the agglutination assay, based on the property of a given molecule to bridge at least two particles.

For diagnosis and quantification of (poly)peptides, polynucleotides, etc. in clinical and/or scientific specimens, a variety of immunological methods, as described above as well as molecular biological methods, like nucleic acid hybridization assays, PCR assays or DNA Enzyme Immunoassays (Mantero et al., Clinical Chemistry 37 (1991), 422-429) have been developed and are well known in the art. In this context, it should be noted that the TIRC7 nucleic acid molecules may also comprise PNAs, modified DNA analogs containing amide backbone linkages. Such PNAs are useful, inter alia, as probes for DNA/RNA hybridization.

The above-described compositions may be used for methods for detecting expression of a TIRC7 polynucleotide by detecting the presence of mRNA coding for a TIRC7 (poly)peptide which comprises, for example, obtaining mRNA from cells of a subject and contacting the mRNA so obtained with a probe/primer comprising a nucleic acid molecule capable of specifically hybridizing with a TIRC7 polynucleotide under suitable hybridization conditions, and detecting the presence of mRNA hybridized to the probe/primer. Further diagnostic methods leading to the detection of nucleic acid molecules in a sample comprise, e.g., polymerase chain reaction (PCR), ligase chain reaction (LCR), Southern blotting in combination with nucleic acid hybridization, comparative genome hybridization (CGH) or representative difference analysis (RDA). These methods for assaying for the presence of nucleic acid molecules are known in the art and can be carried out without any undue experimentation.

Preferably, the sample to be analyzed in accordance with the above described methods are derived from tumor cells or endothelial cells in a tumor. In addition, said metastatic disease is preferably a tumor selected from a group consisting of glioblastoma, medulloblastoma, astrocytoma, primitive neuroectoderma, brain stem glioma cancers, colon carcinoma, bronchial carcinoma, squamous carcinoma, sarcoma, carcinoma in the breast, carcinoma in the head/neck, T cell lymphoma, B cell lymphoma, mesothelioma, leukemia and meningeoma.

The present invention also relates to a kit for use in any one of the above described methods, said kit comprising an anti-TIRC7 antibody or TIRC7 antisense nucleic acid molecule, or a derivative thereof. Such kits are used to detect DNA which hybridizes to TIRC7 DNA or to detect the presence of TIRC7 protein or peptide fragments in a sample. Such characterization is useful for a variety of purposes including but not limited to forensic analyses, diagnostic applications, and epidemiological studies in accordance with the above-described methods of the present invention. The recombinant TIRC7 proteins, DNA molecules, RNA molecules and antibodies lend themselves to the formulation of kits suitable for the detection and typing of TIRC7. Such a kit would typically comprise a compartmentalized carrier suitable to hold in close confinement at least one container. The carrier would further comprise reagents such as recombinant TIRC7 protein or anti-TIRC7 antibodies suitable for detecting TIRC7. The carrier may also contain a means for detection such as labeled antigen or enzyme substrates or the like.

To summarize, the present invention relates to the use of T-cell immune response cDNA 7 (TIRC7) or a fragment thereof, its encoding or regulatory nucleic acid sequences, an activator or antagonist of TIRC7 or a nucleic acid molecule encoding said activator or antagonist in angiogenic or anti-angiogenic therapy. The present invention includes methods of inhibiting tumor growth, comprising administering to the subject in need of such an inhibition a therapeutically effective amount of an antagonist of TIRC7 as defined above, and generally methods for modulating angiogenesis and/or neovascularization, which comprise administering to a subject a therapeutically effective amount of TIRC7 or an activator or antagonist thereof. Preferably, the uses and methods of the present invention are applied to subject suffering from any one of the above described diseases such as solid tumors, developing metastases, ischemic heart diseases and diabetic retinopathy.

In summary, the examples demonstrate that TIRC7 is highly expressed in metastatic tumors and/or their proliferating capillary endothelial cells. It is therefore expected that TIRC7 has a potential in regulation of endothelial cell proliferation and is therefore involved in angiogenesis and neovascularization. This knowledge can be used for the treatment of, for example, angiogenic diseases such as arterial occlusive diseases, like ischemic heart disease. In addition, further experiments demonstrated that anti-TIRC7 antibody has an inhibitory effect on the proliferation of tumor cell lines such as leukemic cell lines for example Jurkat and Sub T1. Since it could be shown that TIRC7 is highly expressed in proliferating capillary endothelial cells and such cells are needed to supply nutrition to tumors, TIRC7 might have a potential in growth inhibition of endothelial cell proliferation and therefore an extremely important function in the treatment and diagnosis of angiogenesis in malignant tumors. Thus, it can be reasonably expected that anti-TIRC7 antibodies as well as other TIRC7 antagonists are able to significantly influence therapeutically, for example, the invasive growth of glioma as well as other tumor cells. Accordingly, the results of the above experiments establish TIRC7 as a novel substance and target in angiogenic and anti-angiogenic therapy.

These and other embodiments are disclosed and encompassed by the description and Examples of the present invention. Further literature concerning any one of the antibodies, methods, uses and compounds to be employed in accordance with the present invention may be retrieved from public libraries and databases, using for example electronic devices. For example the public database “Medline” may be utilized which is available on the Internet, for example under http://www.ncbi.nlm.nih.gov/PubMed/medline.html. Further databases and addresses, such as http://www.ncbi.nlm.nih.gov/, http://www.infobiogen.fr/, http://www.fmi.ch/biology/research_tools.html, http://www.tigr.org/, are known to the person skilled in the art and can also be obtained using, e.g., http://www.lycos.com. An overview of patent information in biotechnology and a survey of relevant sources of patent information useful for retrospective searching and for current awareness is given in Berks, TIBTECH 12 (1994), 352-364.

The use and methods of the invention can be used for the treatment of all kinds of diseases hitherto unknown as being related to or dependent on the modulation of neovascularization, angiogenesis and/or the growth of collateral arteries and/or other arteries from preexisting arteriolar connections. The methods and uses of the present invention may be desirably employed in humans, although animal treatment is also encompassed by the methods and uses described herein.

1. Use of T-cell immune response cDNA 7 (TIRC7), an agent which modulates the activity of TIRC7 or a nucleic acid molecule encoding said. TIRC7 or said agent for the preparation of pharmaceutical compositions for enhancing or suppressing angiogenesis and/or neovascularization.

2. Use of T-cell immune response cDNA 7 (TIRC7), an activator of TIRC7 or of a nucleic acid molecule encoding said TIRC7 or said activator for the preparation of a pharmaceutical composition for enhancing angiogenesis and/or neovascularization

3. The use of claim 2, wherein said pharmaceutical composition is applied to a subject suffering from a vascular disease or a cardiac infarct or a stroke.

4. Use of an antagonist of T-cell immune response cDNA 7 (TIRC7) or of a nucleic acid molecule encoding said antagonist for the preparation of a pharmaceutical composition for suppressing angiogenesis and/or neovascularization.

5. The use of claim 4, wherein said pharmaceutical composition is applied to a subject suffering from a malignant tumor.

6. The use of claim 4, wherein the antagonist is
(i) an anti-TIRC7 antibody or an anti-TIRC7-ligand antibody; and/or
(ii) a non-stimulatory form of TIRC7 or a soluble form of TIRC7 or TIRC7-ligand.

7. Use of anti-TIRC7 antibody or equivalent TIRC7 binding molecule for targeting malignant tumor cells or endothelial cells in malignant tumors.

8. A method of diagnosing a metastatic disease in a subject comprising:
a) assaying a sample from a subject for TIRC7 transcriptional activity or TIRC7 protein; and
b) determining the existence of metastatic disease, wherein the induction of TIRC7 transcriptional activity or the abnormal presence of TIRC7 protein indicates the presence of a metastatic disease.

9. A kit for use in a method of claim 8, said kit comprising an anti-TIRC7 antibody or TIRC7 antisense nucleic acid molecule, or a derivative thereof.

10. A method of angiogenic or anti-angiogenic therapy comprising administering to a subject in need thereof a therapeutically effective amount of TIRC7-cell immune response cDNA 7 (TIRC7) or a fragment thereof, its encoding or regulatory nucleic acid sequences, an activator or antagonist of TIRC7 or a nucleic acid molecule encoding said activator or antagonist.
